(19) United States
(12) Patent Application Publication
(10) Pub. No.: US 2008/0311087 A1
(43) Pub. Date: Dec. 18, 2008

(54) Human Umbilical Tissue-Derived Cell Compositions for the Treatment of Incontinence

(76) Inventors: Anna Gosiewska, Skillman, NJ (US); Agnieszka Seyda, Edison, NJ (US); Charito S. Buensuceso, North Brunswick, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/133,792

(22) Filed: Jun. 5, 2008

Related U.S. Application Data
(60) Provisional application No. 60/944,269, filed on Jun. 15, 2007.

(51) Int. Cl. A61K 35/44; A61P 13/00

(52) U.S. Cl. 424/93.7

(57) ABSTRACT

Compositions for the treatment of incontinence are disclosed. More particularly, compositions of human umbilical tissue-derived cells and a carrier are disclosed. The compositions are useful in the treatment urinary and fecal incontinence.

### FIELD OF THE INVENTION

The invention relates to compositions for the treatment of incontinence. More specifically, the invention relates to compositions comprising cells derived from human umbilical tissue and a carrier for the treatment of incontinence.

### BACKGROUND OF THE INVENTION

Injuries to soft tissue, for example, vascular, skin, or musculoskeletal tissue, are quite common. Many of these disorders occur in the absence of systemic disease and are a consequence of chronic repetitive low-grade trauma and overuse.

One example of a fairly common soft tissue injury is incontinence. Incontinence is the complaint of any involuntary leakage of urine or feces. It can cause embarrassment and lead to social isolation, depression, loss of quality of life, and is a major cause for institutionalization in the elderly population. There are several types of incontinences including urge incontinence or urge urinary incontinence, stress incontinence or stress urinary incontinence, overflow incontinence, and mixed incontinence or mixed urinary incontinence. Mixed incontinence or mixed urinary incontinence refers to the case when a patient suffers from more than one form of urinary incontinence, e.g. stress incontinence and urge incontinence.

The medical need is high for effective pharmacological treatments especially for mixed incontinence and stress urinary incontinence (SUI). This high medical need is a result of lack of efficacious pharmacological therapy coupled with high patient numbers. Recent estimates put the number of people suffering from SUI in the USA at 18 million, with women predominantly affected.

Stress incontinence may be confirmed by observing urine loss coincident with an increase in abdominal pressure, in the absence of a bladder contraction or an overdistended bladder. The condition of stress incontinence may be classified as either urethral hypermobility or intrinsic sphincter deficiency. In urethral hypermobility, the bladder neck and urethra descend during cough or strain and the urethra opens with visible urinary leakage (leak point pressure between 60-120 cm H₂O). In intrinsic sphincter deficiency, the bladder neck opens during bladder filling without bladder contraction. Visible urinary leakage is seen with minimal or no stress. There is variable bladder neck and urethral descent, often none at all, and the leak point pressure is low (<60 cm H₂O). (J. G. Blaivas, 1985, Urol. Clin. N. Amer., 12:215-224; D. R. Staskin et al., 1985, Urol. Clin. N. Amer., 12:271-278).

Urge incontinence is defined as the involuntary loss of urine associated with an abrupt and strong desire to void. Although involuntary bladder contractions can be associated with neurologic disorders, they can also occur in individuals who appear to be neurologically normal (P. Abrams et al., 1987, Neurol. & Urodynam., 7:403-427). Common neurologic disorders associated with urge incontinence are stroke, diabetes, and multiple sclerosis (E. J. McGuire et al, 1981, J. Urol., 126:205-209). Urge incontinence is caused by involuntary detrusor contractions that can also be due to bladder inflammation and impaired detrusor contractility where the bladder does not empty completely.

Overflow incontinence is characterized by the loss of urine associated with overdistension of the bladder. Overflow incontinence may be due to impaired bladder contractility or to bladder outlet obstruction leading to overdistension and overflow. The bladder may be underactive secondarily to neurologic conditions such as diabetes or spinal cord injury, or following radical pelvic surgery.

Another common and serious cause of urinary incontinence (urge and overflow type) is impaired bladder contractility. This is an increasingly common condition in the geriatric population and in patients with neurological diseases, especially diabetes mellitus (N. M. Resnick et al., 1989, New Engl. J. Med., 320:1-7; M. B. Chancellor and J. G. Blaivas, 1996, Atlas of Urodynamics, Williams and Wilkins, Philadelphia, Pa.). With inadequate contractility, the bladder cannot empty its content of urine; this causes not only incontinence, but also urinary tract infection and renal insufficiency. Presently, clinicians are very limited in their ability to treat impaired detrusor contractility. There are no effective medications to improve detrusor contractility. Although urecholine can slightly increase intravesical pressure, it has not been shown in controlled studies to aid effective bladder emptying (A. Wein et al., 1980, J. Urol., 123:302). The most common treatment is to circumvent the problem with intermittent or indwelling catheterization.

There are a number of treatment modalities for stress urinary incontinence. The most commonly practiced current treatments for stress incontinence include the following: absorbent products; indwelling catheterization; pessary, i.e., vaginal ring placed to support the bladder neck; and medication (Agency for Health Care Policy and Research. Public Health Service: Urinary Incontinence Guideline Panel. Urinary Incontinence in Adults: Clinical Practice Guideline. AHCPR Pub. No. 92-0038. Rockville, Md. U.S. Department of Health and Human Services, March 1992; M. B. Chancellor, Evaluation and Outcome. In: The Health of Women With Physical Disabilities: Setting a Research Agenda for the 90's. Eds. Krotoski D. M., Nosek, M., Turk, M., Brooks Publishing Company, Baltimore, Md., Chapter 24, 309-332, 1996). Exercise is another treatment modality for stress urinary incontinence. For example, Kegel exercise is a common and popular method to treat stress incontinence. The exercise can help half of the people who can do it four times daily for 3-6 months. Although 50% of patients report some improvement with Kegel exercise, the cure rate for incontinence following Kegel exercise is only 5 percent. In addition, most patients stop the exercise and drop out from the protocol because of the very long time and daily discipline required.

Another treatment method for urinary incontinence is the urethral plug. This is a disposable cork-like plug for women with stress incontinence. Unfortunately, the plug is associated with over 20% urinary tract infection and, unfortunately, does not cure incontinence.

Biofeedback and functional electrical stimulation using a vaginal probe are also used to treat urge and stress urinary incontinence. However, these methods are time-consuming and expensive and the results are only moderately better than Kegel exercise. Surgeries, such as laparoscopic or open abdominal bladder neck suspensions; transvaginal approach abdominal bladder neck suspensions; artificial urinary sphincter (expensive complex surgical procedure with 40% reversion rate) are also used to treat stress urinary incontinence.

Other treatments include intra-urethral injection procedures with exogenous injectable materials such as silicone, carbon-coated particles, Teflon, collagen, and autologous fat. Each of these injectables has its disadvantages. U.S. Pat. Nos. 5,007,940; 5,158,573; and 5,116,387 to Berg report biocompatible compositions comprising discrete, polymeric and silicone rubber bodies injectable into urethral tissue for the purpose of treatment of urinary incontinence by tissue bulking. Further, U.S. Pat. No. 5,451,406 to Lawin reports biocompatible compositions comprising carbon coated particulate substrates that may be injected into a tissue, such as the tissues of and that overlay the urethra and bladder neck, for the purpose of treatment of urinary incontinence by tissue bulking. One concern or adverse consequence associated with methodologies or therapies of tissue bulking relates to the migration of solid particles in the bulking agents from the original site of placement into repository sites in various body organs and the subsequent chronic inflammatory response of tissue to particles that are too small. These adverse effects are reported in urology literature, specifically in Malizia, A. A., et al., “Migration and Granulomatous Reaction After Periurethral Injection of Polytef (Teflon),” JAMA, 251:3277-3281 (1984) and in Claes, H., Stroobants, D. et al., “Pulmonary Migration Following Periurethral Polytetrafluoroethylene Injection For Urinary Incontinence,” J. Urol., 142:821-822 (1989). An important factor in assuring the absence of migration is the administration of properly sized particles. If particles are too small, they may be engulfed by the body's white cells (phagocytes) and carried to distant organs or may be carried away in the vascular system and travel until they reach a site of greater constriction. Target organs for particulate deposition include the lungs, liver, spleen, brain, kidney, and lymph nodes. The use of small diameter particulate spheres and elongate fibrils in an aqueous medium having biocompatible lubricant have been disclosed in Wallace et al., U.S. Pat. No. 4,803,075. While these materials showed positive, short-term augmentation results, these results were short-lived as the material had a tendency to migrate and/or be absorbed by the host tissue.

Collagen injections generally employ bovine collagen, which absorbs in 4-6 months, resulting in the need for repeated injections. A further disadvantage of collagen is that about 5% of patients are allergic to bovine source collagen and develop antibodies.

Autologous fat grafting as an injectable bulking agent has a significant drawback in that most of the injected fat is resorbed. In addition, the extent and duration of the survival of an autologous fat graft remains controversial. An inflammatory reaction generally occurs at the site of implant. Complications from fat grafting include fat resorption, nodules and tissue asymmetry.

Recent approaches with muscle cell injection therapy using engineered muscle-derived cells might offer alternative therapy for the treatment of incontinence, particularly, stress urinary incontinence and for the enhancement of urinary continence. Preferably, the muscle-derived cell injection can be autologous, so that there will be minimal or no allergic reactions. Myoblasts, the precursors of muscle fibers, are mononucleated muscle cells, which differ in many ways from other types of cells. Myoblasts naturally fuse to form post-mitotic multinucleated myotubes which result in the long-term expression and delivery of bioactive proteins (T. A. Partridge and K. E. Davies, 1995, Brit. Med. Bulletin, 51:123-137; J. Dhawan et al., 1992, Science, 254: 1509-1512; A. D. Grinnell, 1994, In: Myolo. Ed 2, Ed. Engel A G and Armstrong C F, McGraw-Hill, Inc, 303-304; S. Jiao and J. A. Wolff, 1992, Brain Research, 575:143-147; H. Vandenburgh, 1996, Human Gene Therapy, 7:2195-2200).

The use of myoblasts to treat muscle degeneration, to repair tissue damage or treat disease is disclosed in U.S. Pat. Nos. 5,130,141 and 5,538,722. Also, myoblast transplantation has been employed for the repair of myocardial dysfunction (S. W. Robinson et al., 1995, Cell Transplantation, 5:77-91; C. E. Murry et al., 1996, J. Clin. Invest., 98:2512-2523; S. Gojo et al., 1996, Cell Transplantation, 5:581-584; A. Zibaitis et al., 1994, Transplantation Proceedings, 26:3294). The use of myoblasts for treating urinary incontinence is disclosed in U.S. Pat. No. 6,866,842. as well as Transplantation. Oct. 15, 2003;76(7):1053-60; J. Urol. January 2001;165(1):271. and Yokoyama T. J,. Urology, 165:271-276, 2001. Application WO2004055174, discloses culture medium composition, culture method, and myoblasts obtained, and their uses. Soft tissue and bone augmentation and bulking utilizing muscle-derived progenitor cells, compositions and treatments is disclosed in WO0178754. Myoblast therapy for mammalian diseases is disclosed in U.S. Pat. No. 9,909,451.

Although, the cell therapy offers advantages over other injectables, it has major disadvantages. One of the biggest limitations associated with the use of myoblasts for the treatment of stress urinary incontinence is that myoblasts require extensive in vitro cultivation for 3-4 weeks to achieve cell numbers required for injection making this therapy very expensive and unaffordable to many patients.

In view of the above-mentioned limitations and complications of treating urinary incontinence and bladder contractility, new and effective alternative modalities in this area are needed in the art.

### SUMMARY OF THE INVENTION

The invention is a composition for the treatment of incontinence comprising cells derived from human umbilical tissue referred to herein as human umbilical tissue-derived cells (hUTC) and a carrier. The composition contains at least one hUTC that can migrate from the carrier and onto the transplantation site to form a new tissue. The hUTC may be obtained from allogeneic tissue. The carrier includes, but is not limited to physiological buffer solution, injectable gel solution, saline and water. The compositions are useful in the treatment of incontinence by injecting the composition into the urogentital tissue, such as urethra, urethral sphincter, and bladder for urinary incontinences and colorectal tissue, such as colon, rectum and colorectal sphincter for fecal incontinence.

### DETAILED DESCRIPTION

The methods for isolating and collecting human umbilical tissue-derived cells (hUTCs) (also referred to as umbilical-derived cells (UDCs)) are described in copending U.S. application Ser. No. 10/877,012 incorporated herein by reference in its entirety. To collect postpartum umbilicus for the isolation and culture of cells the umbilicus is obtained immediately post childbirth. For example, but not by way of limitation, following removal of the umbilical cord (drained of blood), or a section thereof, may be transported from the birth site to the laboratory in a sterile container such as a flask, beaker or culture dish, containing a salt solution or medium, such as, for example, Dulbecco's Modified Eagle's Medium (DMEM). The umbilical cord is preferably maintained and handled under sterile conditions prior to and during collection of the tissue, and may additionally be surface-sterilized by brief surface treatment of the cord with, for example, a 70 percent by volume ethanol in water solution, followed by a rinse with sterile, distilled water or isotonic salt solution. The umbilical cord can be briefly stored for about 1 to 24 hours at about 3° to about 50° C. It is preferable to keep the tissue at 4° to 10° C., but not frozen, prior to extraction of cells. Antibiotic or antimycotics may be included in the medium to reduce microbiological contamination. Cells are collected from the umbilical cord under sterile conditions by any appropriate method known in the art. These examples include digestion with enzymes such as dispase, collagenase, trypsin, hyaluronidase, or dissection or mincing. Isolated cells or tissue pieces from which cells grow out may be used to initiate cell cultures.

The umbilical tissue may be rinsed with anticoagulant solution such as heparin. The tissue may be transported in solutions used for tranportation of organs used for transplantation such as University of Wisconsin solution or Perfluorochemical solution.

Isolated cells are transferred to sterile tissue culture vessels either uncoated or coated with extracellular matrix or ligands such as laminin, collagen, gelatin. To grow the cells culture media is added such as, DMEM (high or low glucose), McCoy's 5A medium, Eagle's basal medium, CMRL medium, Glasgow minimum essential medium, Ham's F-12 medium (F12), Iscove's modified Dulbecco's medium, Liebovitz L-15 medium, MCDB, and RPMI 1640, among others. The culture medium may be supplemented with one or more components including, for example, fetal bovine serum (FBS), equine serum (ES), human serum (HS), growth factors, for example PDGF, FGF, erythropoietin and one or more antibiotics and/or antimycotics to control microbial contamination, such as, penicillin G, streptomycin sulfate, amphotericin B, gentamicin, and nystatin, either alone or in combination, among others.

The cells in culture vessels at a density to allow cell growth are placed in an incubator with 0 to 5 percent by volume CO₂ in air and 2 to 25 percent O₂ in air at 25 to 40° C. The medium in the culture vessel can be static or agitated, for example using a bioreactor. Cells may be grown under low oxidative stress (e.g. with addition of glutathione, Vitamin C, Catalase, Vitamin E, N-Acetylacysteine). “Low oxidative stress”, as used herein, refers to conditions of no or minimal free radical damage to the cultured cells. Cells may also be grown under alternating conditions, for example, in a period of normoxia followed by a period of hypoxia.

Methods for the selection of the most appropriate culture medium, medium preparation, and cell culture techniques are well known in the art and are described in a variety of sources, including Doyle et al., (eds.), 1995, Cell & Tissue Culture: Laboratory Procedures, John Wiley & Sons, Chichester; and Ho and Wang (eds.), 1991, Animal Cell Bioreactors, Butterworth-Heinemann, Boston, which are incorporated herein by reference in their entirety.

After culturing the isolated cells or tissue pieces for a sufficient period of time, for example, about 10 to about 12 days, umbilical cells present in the explanted tissue will tend to have grown out from the tissue, either as a result of migration there from or cell division, or both. Umbilical cells may then be removed to a separate culture vessel containing fresh medium of the same or a different type as that used initially, where the population of cells can be mitotically expanded.

Alternatively, the cells present in postpartum tissue can be fractionated into subpopulations from which the postpartum cells can be isolated. This may be accomplished using standard techniques for cell separation including, but not limited to, enzymatic treatment to dissociate postpartum tissue into its component cells, followed by cloning and selection of specific cell types, using either morphological or biochemical markers, selective destruction of unwanted cells (negative selection), separation based upon differential cell agglutinability in the mixed population as, for example, with soybean agglutinin, freeze-thaw procedures, differential adherence properties of the cells in the mixed population, filtration, conventional and zonal centrifugation, centrifugal clutriation (counter-streaming centrifugation), unit gravity separation, countercurrent distribution, electrophoresis, and fluorescence activated cell sorting (FACS). For a review of clonal selection and cell separation techniques, see Freshney, 1994, Culture of Animal Cells; A Manual of Basic Techniques, 3rd Ed., Wiley-Liss, Inc., New York, which is incorporated herein by reference in its entirety.

The medium is changed as necessary by carefully aspirating the medium from the dish, for example, with a pipette, and replenishing with fresh medium. Incubation is continued as described above until a sufficient number or density of cells accumulate in the dish, for example, approximately 70 percent confluence. The original explanted tissue sections may be removed and the remaining cells are trypsinized using standard techniques or using a cell scraper. After trypsinization, the cells are collected, removed to fresh medium and incubated as described above. The medium may be changed at least once at 24 hours post-trypsin to remove any floating cells. The cells remaining in culture are umbilical tissue-derived cells.

Umbilical tissue-derived cells can be characterized using flow cytometry, immunohistochemistry, gene arrays, PCR, protein arrays or other methods known in the art.

Umbilical tissue-derived cells can undergo at least 10 population doublings. One of skill in the art would be able to determine when a cell has undergone a population doubling (Freshney, R. I. Culture of Animal Cells: A Manual of Basic 15 Techniques New York, Wiley-Liss 1994).

While an umbilical tissue-derived cell can be isolated, preferably it is within a population of cells. The invention provides a defined population of umbilical tissue-derived cells. In one embodiment, the population is heterogeneous. In another embodiment, the population is homogeneous.

The umbilical tissue-derived cells have been phenotypically characterized for one or more of the markers CD10, CD13, CD31, CD34, CD44, CD45, CD73, CD90, CD117, CD141, PDGFr-α, HLA-A, HLA-B, HLA-C, HLA-DR, HLA-DP, and HLA-DQ. In one embodiment, the hUTC have been characterized as having a phenotype comprising CD10+, CD13+, CD31−, CD34−, CD44+, CD45−, CD73+, CD90+, CD117−, CD141−, PDGFr-α+, HLA-A+, HLA-B+, HLA-C+, HLA-DR−, HLA-DP−, and HLA-DQ− and telomerase-. In another embodiment, the hUTCs are phenotypically CD13+, CD90+, CD34−, and CD117−. In yet another embodiment, the hUTC are phenotypically CD10+, CD13+, CD44+, CD73+, CD90+ PDGFr-α+, PD-L2+, HLA-A+, HLA-B+, HLA-C+, and CD31−, CD34− CD45−, CD80−, CD86−, CD117−, CD141−, CD178−, B7-H2−, HLA-G−, HLA-DR−, HLA-DP−, and HLA-DQ−.

hUTC express several neurotrophic factors including MCP-1, IL-6, IL-8, GCP-2, HGF, FGF, HB-EGF, BDNF, TPO, MIP1a, RANTES, and TIMP1 suggesting the ability to provide trophic support to cells of a soft tissue phenotype. Conversely, these cells lack of secretion of at least one of TGF-beta2, ANG2, PDGFbb, MIP1b, 1309, MDC, and VEGF.

The composition of the present invention also includes a carrier. The carrier is biocompatible, easily sterilized and has sufficient physical properties to provide for ease of injection. The carrier includes, but is not limited to physiological buffer solution, injectable gel solution, saline and water. Physiological buffer solution includes, but is not limited to buffered saline, phosphate buffer solution, Hank's balanced salts solution, Tris buffered saline, and Hepes buffered saline. In one embodiment, the physiological buffer is Hank's balanced salts solution. The injectable gel solution may be in a gel form prior to injection or may gel and stay in place upon administration.

The injectable gel solution is comprised of water, saline or physiological buffer solution and a gelling material. Gelling materials include, but are not limited to proteins such as, collagen, elastin, thrombin, fibronectin, gelatin, fibrin, tropoelastin, polypeptides, laminin, proteoglycans, fibrin glue, fibrin clot, platelet rich plasma (PRP) clot, platelet poor plasma (PPP) clot, self-assembling peptide hydrogels, and atelocollagen; polysaccharides such as, pectin, cellulose, oxidized cellulose, chitin, chitosan, agarose, hyaluronic acid; polynucleotides such as, ribonucleic acids, deoxyribonucleic acids, and others such as, alginate, cross-linked alginate, poly(N-isopropylacrylamide), poly(oxyalkylene), copolymers of poly(ethylene oxide)-poly(propylene oxide), poly(vinyl alcohol), polyacrylate, monostearoyl glycerol co-Succinate/polyethylene glycol (MGSA/PEG) copolymers and combinations thereof.

In one embodiment, the composition further comprises microparticles. Microparticles are also referred to as microbeads or microspheres by one of skill in the art. The microparticles provide both a temporary bulking effect and a substrate on which the viable muscle tissue fragments may adhere and grow. The microparticles must be large enough so as to discourage local and distant migration once injected, yet small enough so as to be administered by a hypodermic needle. Thus, microparticles have a substantially round shape with an average transverse cross-sectional dimension in the range of about 100 to about 1,000 microns, preferably in the range of about 200 to about 500 microns. The microparticles are preferably formed from a biocompatible polymer. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring. The biocompatible polymers may also be biodegradable. Biodegradable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

In one embodiment, the microparticle is comprised of at least one synthetic polymer. Suitable biocompatible synthetic polymers include, but are not limited to polymers of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof. Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of monomers including lactide (which includes lactic acid, D-, L- and meso lactide); glycolide (including glycolic acid); epsilon-caprolactone; p-dioxanone(1,4-dioxan-2-one); trimethylene carbonate(1,3-dioxan-2-one); alkyl derivatives of trimethylene carbonate; and blends thereof. Aliphatic polyesters used in the present invention can be homopolymers or copolymers (random, block, segmented, tapered blocks, graft, triblock, etc.) having a linear, branched or star structure.

In embodiments where the scaffold includes at least one natural polymer, suitable examples of natural polymers include, but are not limited to, fibrin-based materials, collagen-based materials, hyaluronic acid-based materials, glycoprotein-based materials, cellulose-based materials, silks and combinations thereof.

One skilled in the art will appreciate that the selection of a suitable material for forming the biocompatible microparticles depends on several factors. These factors include in vivo mechanical performance; cell response to the material in terms of cell attachment, proliferation, migration and differentiation; and optionally, biodegradation kinetics. Other relevant factors include the chemical composition, spatial distribution of the constituents, the molecular weight of the polymer, and the degree of crystallinity.

In another embodiment, a biological effector may be incorporated within the composition of the invention. The biological effectors, promote the healing and/or regeneration of the affected tissue (e.g. growth factors and cytokines), prevent infection (e.g., antimicrobial agents and antibiotics), reduce inflammation (e.g., anti-inflammatory agents), prevent or minimize adhesion formation, such as oxidized regenerated cellulose (e.g., INTERCEED and Surgicel®, available from Ethicon, Inc.) and hyaluronic acid, and suppress the immune system (e.g., immunosuppressants).

Biological effectors include, but are not limited to heterologous or autologous growth factors, matrix proteins, peptides, antibodies, enzymes, glycoproteins, hormones, cytokines, glycosaminoglycans, nucleic acids, analgesics. It is understood that one or more biological effectors of the same or different functionality may be incorporated within the composition.

Heterologous or autologous growth factors are known to promote healing and/or regeneration of injured or damaged tissue. Exemplary growth factors include, but are not limited to, TGF-β, bone morphogenic protein, growth differentiation factor-5 (GDF-5), cartilage-derived morphogenic protein, fibroblast growth factor, platelet-derived growth factor, vascular endothelial cell-derived growth factor (VEGF), epidermal growth factor, insulin-like growth factor, hepatocyte growth factor, and fragments thereof. Suitable effectors likewise include the agonists and antagonists of the agents noted above.

Glycosaminoglycans are highly charged polysaccharides, which play a role in cellular adhesion. Exemplary glycosaminoglycans useful as biological effectors include, but are not limited to heparin sulfate, heparin, chondroitin sulfate, dermatan sulfate, keratin sulfate, hyaluronan (also known as hyaluronic acid), and combinations thereof.

The biological effector may also be an enzyme such as, matrix-digesting enzymes, which facilitate cell migration out of the extracellular matrix surrounding the cells. Suitable matrix-digesting enzymes include, but are not limited to collagenase, chondroitinase, trypsin, elastase, hyaluronidase, peptidase, thermolysin, matrix metalloprotease and protease.

One of ordinary skill in the art will appreciate that the appropriate biological effector(s) may be determined by a surgeon, based on principles of medical science and the applicable treatment objectives. The amount of the biological effector included with the composition will vary depending on a variety of factors, including the given application, such as promoting cell survival, proliferation, differentiation, or facilitating and/or expediting the healing of tissue. The biological effector can be incorporated within the composition of viable muscle tissue fragments and carrier before or after the composition is administered to the area of tissue injury.

The composition for treating incontinence as described herein may be prepared by first obtaining allogeneic hUTC via the methods described above. The hUTC are combined with a carrier, as described herein, and optionally with microparticles and delivered to the site of tissue repair via injection. In addition, a biological effector may be added to the composition with or without microparticles prior to administration to the site of tissue repair.

A kit can be used to assist in the preparation of the compositions. The kit includes a sterile container that houses a reagent for sustaining cell viability, a carrier, and a delivery device. The cells may be placed in the sterile container containing the reagent for sustaining viability. Suitable reagents for sustaining the viability of the include but are not limited to saline, phosphate buffering solution, Hank's balanced salts, standard cell culture medium, Dulbecco's modified Eagle's medium, ascorbic acid, HEPES, nonessential amino acid, L-proline, autologous serum, and combinations thereof. The carrier may be physiological buffer solution, injectable gel solution, saline or water as described herein and may optionally include microparticles. The delivery device allows deposition of the composition in a carrier into diseased tissues, for example adjacent to or surrounding the sphincter regions of the urethra.

Compositions as described herein are useful in the treatment of soft tissue. Soft tissue refers generally to extraskeletal structures found throughout the body and includes but is not limited to, periodontal tissue, skin tissue, vascular tissue, muscle tissue, fascia tissue, ocular tissue, pericardial tissue, lung tissue, synovial tissue, nerve tissue, kidney tissue, esophageal tissue, urogenital tissue, intestinal tissue, colorectal tissue, liver tissue, pancreas tissue, spleen tissue, adipose tissue, and combinations thereof. Preferably, the compositions as described herein are useful in the treatment of urogenital tissue, such as urethra, urethral sphincter, and bladder, esophageal tissue, such as esophagus and esophageal sphincter, and colorectal tissue, such as colon, rectum and colorectal sphincter. The compositions can also be used for tissue bulking, tissue augmentation, cosmetic treatments, therapeutic treatments, and for tissue sealing.

### EXAMPLE 1

The efficacy of a novel therapy based on the application of a composition of hUTC for the restoration of leak point pressure (LPP) in a rat model of stress urinary incontinence (SUI) was examined. hUTC were thawed from liquid nitrogen. A total of 24 female Lewis rats were randomly assigned to 1 of 3 groups (8 animals per group), namely continent animals, incontinent animals injected with carrier, and incontinent animals injected with carrier +hUTC. SUI was created in the latter 2 groups by bilateral pudendal nerve transection (PNT). One week post-surgery, treatment was administered to each animal group by an intraurethral injection. After 5 weeks LPP was measured 5 or 6 times in each rat and the mean was determined.

### Animal Care

The animals used in this study were handled and maintained in accordance with all applicable sections of the Final Rules of the Animal Welfare Act regulations (9 CFR), the Public Health Service Policy on Humane Care and Use of Laboratory Animals, the Guide for the Care and Use of Laboratory Animals. The protocol and any amendments or procedures involving the care or use of animals in this study was reviewed and approved by the Testing Facility Institutional Animal Care and Use Committee prior to the initiation of such procedures.

Lewis rats were chosen due to their syngeneic phenotype. It allows evaluation of a composition for treatment of SUI derived from one rat and implanted into another without the use of immunosupression. The animals were individually housed in microisolators. Environmental controls were set to maintain temperatures of 18° C. to 26° C. (64° F. to 79° F.) with a relative humidity of 30% to 70%. A 12-hour light/12-hour dark cycle was maintained, except when interrupted to accommodate study procedures. Ten or greater air changes per hour with 100% fresh air (no air recirculation) was maintained in the animal rooms. Purina Certified Diet and filtered tap water was provided to the animals ad libitum.

### Materials and Methods

Animals. SUI was created by the previously established method of bilateral pudendal nerve transection (PNT). All procedures were performed under aseptic conditions. The rats were prepared for aseptic surgery and anesthesia was induced using isoflurane at 2.5%-4%. After induction, anesthesia was maintained with isoflurane delivered through a nose cone at 0.5-2.5%. For PNT surgery, the hair over the region spanning from the hips to the base of the tail, over the rump and down the back of the hind legs was shaved and the animal positioned in ventral recumbency. Via a dorsal longitudinal incision, the ischiorectal fossa was opened bilaterally. Using loop magnification the pudendal nerve was isolated and transected. The incision was closed using Nexaband® liquid topical tissue adhesive. The continent animal group had undergone the same surgical procedure with the exception of actually transecting the nerve.

Composition preparation and administration. hUTC (isolated as described in U.S. Application Publication No. 20050054098 A1, Example 1) were thawed from liquid nitrogen. Cells were removed from liquid nitrogen and rapidly thawed in a 37° C. water bath with gentle swirling. The contents of the vials was transferred to a 15 mL centrifuge tube containing HBSS. Cells were centrifuged at 150×g for 5 min at 4° C. in a clinical centrifuge. The supernatant was gently aspirated and cells were resuspended in 5 mL of HBSS by gentle pipetting. Cells were placed on ice and counted with a hemocytometer. Cells were spun down and resuspended in HBSS at 1.5×10⁶ cells per 20 microliters. The hUTC suspended in HBSS were loaded into a 100 microliter Hamilton syringe and injected into the rat urethra with a hypodermic needle. Animals underwent treatment one-week post SUI injury creation. The female rats were anesthetized and then two injections (10 microliters each) per rat were performed at the 2-o'clock and 10-o'clock positions of the urethra. The carrier treated animals received injections of HBSS alone in the same manner.

Leak Point Pressure (LPP) Testing. At 5 weeks post-surgery, the rats were anesthetized and placed supine at the level of zero pressure and the bladder emptied manually. Subsequently the bladder was filled with saline solution at room temperature (5 ml per hour) through a suprapubic catheter. The suprapubic catheter was connected to a syringe pump and a pressure transducer. All bladder pressures were referenced to air pressure at bladder level. Pressure and force transducer signals were amplified and digitized for computer data collection using AD instruments, Power Lab computer software at 10 samples per second.

Peak bladder pressure was generated by slowly and manually increasing abdominal pressure until a leak occurred, at which point external abdominal pressure was rapidly released. LPP testing was performed a minimum of four times in each rat. The bladder was emptied using the Credé maneuver and refilled between LPP measurements. LPP values were acquired using an AD Instruments pressure transducer and analyzed using Power Lab Chart™ computer software. Individual outliers within LPP testing sessions for each animal were qualitatively identified as pressure artifacts and excluded from the study. Artifact pressure results were defined as pressure values (mmHg) that were considered artificially high or low compared to the other pressure results from the same LPP testing session. During LPP testing pressure artifacts can be generated in multiple ways including; inadvertently obstructing the catheter tip against either the mucosal wall of the bladder or urethra, the bladder not being completely evacuated of urine and/or saline, the animal being light on anesthetics during testing resulting in the animal contracting its bladder.

### Results and Discussion

The average LPP and standard deviation are reported below.

| Treatment | Number of | Average LPP | Standard |
|---|---|---|---|
| Group | animals | (mm Hg) | Deviation |
| Continent | 4 | 42.6 | 5.4 |
| animals | | | |
| Incontinent | 8 | 22.9 | 3.1 |
| animals injected | | | |
| with carrier | | | |
| Incontinent | 8 | 34.5 | 3.1 |
| animals injected | | | |
| with carrier + | | | |
| hUTC | | | |

### Conclusions

The data indicates that functional improvement was observed after four weeks in incontinent animals treated with hUTC as compared to the incontinent animals injected with carrier alone. The improvement achieved was approximately 81% of continent animals, which indicates 55% improvement over incontinent animals injected with carrier alone. The data indicates that hUTC produced a visible improvement over vehicle therapy alone and therefore can be a therapy for the treatment of stress urinary incontinence.

### EXAMPLE 2

The efficacy of a novel therapy based on the application of a composition of hUTC for the restoration of leak point pressure (LPP) in 2 rat models of stress urinary incontinence (SUI) can be examined side by side. hUTC are thawed from liquid nitrogen. The 2 different rat models that can be compared are incontinent animals resulting from bilateral pudendal nerve transsection and from urethrolysis. Urethrolysis model will be created by a previously established method. Briefly, the animals will be anesthetized with an intraperitoneal injection of ketamine (60 mg/kg body wt) and xylazine (5 mg/kg body wt). They will be placed supine on a water-circulating heating pad. The abdomen will be prepped and draped in standard surgical fashion. A lower abdominal midline incision will be made, and the bladder and urethra will be identified. The proximal and distal urethra will be detached circumferentially by incising the endopelvic fascia and detaching the urethra from the anterior vaginal wall and pubic bone by sharp dissection. Care will be taken not to injure the ureters or compromise the inferior vesical vasculature. A cotton swab will be put into the vagina to aid with the dissection. The rectus fascia and skin will be closed with 4-0 polyglactin (Vicryl) and 4-0 Nylon sutures, respectively.

There will be 3 groups per injury model and rats can be randomly assigned to 1 of 3 groups namely continent animals, incontinent animals injected with carrier, and incontinent animals injected with carrier+hUTC. One week post-surgery, treatment can be administered to each animal group by an intraurethral injection. After 5 weeks LPP can be measured 5 or 6 times in each rat and the mean can be determined.

### EXAMPLE 3

Description of various routes of administration of the composition into the urethra.

Periurethral route of minced tissue injection. Dispense the hUTC composition containing microparticles into the special high-pressure syringe connected to a 17-gauge needle. Slowly insert the needle next to the urethral opening and into the submucosal tissues. After ascertaining the proper position of the needle, inject the suspension at 3 places around the urethra: the 2-, 6-, and 10-o'clock positions. As the injection progresses, the urethral lumen can be observed closing, and then the opening disappears. To assure success, visualize complete apposition (ie, kissing) of the urethral mucosa at the end of the procedure. One or 2 tubes may be injected to produce complete closure of the urethra.

Transurethral route. Using a special needle, inject hUTC composition under direct vision underneath the urethral mucosa. Insert the cystoscope into the mid urethra. Under cystoscopic vision, carefully insert the tip of the needle underneath the urethral mucosa. Precisely deposit the hUTC into the submucosal tissues until complete coaptation of the urethral mucosa is visualized.

Antegrade route. The antegrade route is reserved for males who are incontinent postprostatectomy. Create a suprapubic tract under adequate anesthesia. General anesthesia is preferred. Insert a flexible cystoscope into the bladder via the suprapubic tract. Identify the bladder neck. Under cystoscopic vision, carefully insert the tip of the needle underneath the bladder neck mucosa. Precisely deposit the hUTC formulation into the submucosal tissues until complete coaptation of the bladder neck is noted.

### EXAMPLE 4

Thaw the hUTC from liquid nitrogen. The hUTC can be combined with a required volume, of carrier such as phosphate buffered saline (PBS) or HBSS or other carrier such as aqueous collagen solution, aqueous hyaluronic acid solution and microcarrier such as poly(glycolic acid) (PGA) orpoly(lactic acid) (PLA). The process of mixing is followed by an immediate injection into the mid-urethra or the bladder neck of incontinent animals. At baseline and 3-4 weeks post-op, all of animals can undergo urodynamic testing. Urethral tissue can be harvested for organ bath isometric studies to test urethral function and for immunochemistry.

### EXAMPLE 5

The objective is to show that in pigs, hUTC can be mixed with a carrier (PBS, HBSS, aqueous collagen solution, aqueous HA solution) and injected under sonographic control into the urethra. In addition, this procedure can be used to evaluate the composition as described herein as a therapeutic approach to treat urinary incontinence especially stress urinary incontinence. The hUTC can be combined with a carrier and/or microparticles. With the help of transurethral ultrasound probe and injection system, samples can be injected into the rhabdosphincter and the urethral submucosa. Urethral pressure profiles can be measured before and after injection to determine the postoperative changes of urethral closure pressures. Histology can also performed on specimen obtained from pigs post-operatively.

### EXAMPLE 6

hUTC can be combined with a required volume of carrier and optionally microparticles as detailed in previous examples and can be injected into the internal or external anal sphincters using techniques known in the art for the treatment of fecal incontinence.

### EXAMPLE 7

hUTC can be combined with a required volume of carrier and optionally microparticles as detailed in previous examples and using techniques known in the art can be injected into the lower esophageal sphincter and or the pyloric sphincter for the treatment of acid reflux and other digestive system related ailments.

### EXAMPLE 8

### Porcine Urethral Cell Isolation

Porcine urethras were procured from Farm-to-Pharm (Warren, N.J.). Urethras were trimmed of fat and connective tissue and finely minced with a pair of scalpels. The weight of tissue was recorded (13.1 g) and tissue was placed in a 50 ml conical tube in a cocktail of digestion enzymes (see below) in DMEM (Invitrogen, Carlsbad, Calif.), 10% FBS (Hyclone, Logan, Utah), penicillin/streptomycin (Invitrogen, Carlsbad, Calif.).

The tube was wrapped with Parafilm M® to seal. The tube was transferred to 37° C. incubator shaking at 225 RPM for 2 hours. The completeness of digestion was checked every hour of incubation by removing the tube from the incubator and stand the tube upright for 1-2 minutes. When digestion was complete (no more than 2 hrs) the tube was stood upright for 1-2 minutes to allow large fragments to settle. The cell suspension (without the large fragments) was transfered to a new conical tube and diluted with fresh DMEM, 10% FBS, penicillin/streptomycin. Cell suspension was centrifuge at 150*g for 5 min and supernatant aspirated. Fresh medium was added (up to 50 ml in total volume) and resuspended. Cell suspension was centrifuge at 150*g for 5 min and supernatant removed. Fresh medium was added (up to 30 ml in total volume) and cells resuspended using a pipette by pipetting up and down. Resuspended cell pellet was filtered through a 100 μm filter. Cell suspension was centrifuged at 150*g for 5 min the supernatant aspirated and cell pellet resuspended in PBS. Cells were counted with the GUAVA® cell counter (Guava Technologies, Inc, Hayward, Calif.). Total of ˜6×10⁶ cells was obtained. Cells were plated in EGM-2 (Lonza, Walkersville, Md.) at 5,000 cells/cm² and placed in an incubator at 37° C.

### Digestion Enzymes

Collagenase 0.25 U/ml (Serva Electrophoresis, GmbH, Heidelberg, Germany), 2.5 U/ml dispase (Dispase II 165859, Ruche Diagnostics Corporation, Indianapolis, Ind.) and 1 U/ml hyaluronidase (Vitrase, ISTA Pharmaceuticals, Irvine, Calif.).

### Proliferation Assay

To assess effect hUTC on the proliferation of cells isolated from porcine urethra. Urethra cells (isolated according to the method described above) were seeded onto 24-well dishes at a density of 10,000 cells/well. Experimental conditions were:
Low serum (please fill in)
Low serum (please fill in)+different amounts of hUTC (6600, 3300, or 1650 and 825 cells/well)
   hUTC were added to the inside of transwells (0.4 micron pore size) in EGM-2/Hayflick (20/80) medium. At 3 and 7 days, urothelial cells were harvested to obtain cell number and viability using the Guava instrument (Guava Technologies, Inc, Calif.).

### Results:

| | Mean + std dev | |
|---|---|---|
| | day 3 | day 7 |
| 20% EGM/Hayflick | 8510 ± 1212 | 10803 + 1064 |
| hUTC (6600) | 9048 ± 962 | 14624 + 2052 |
| hUTC (3300) | 6410 ± 703 | 10673 + 1794 |
| hUTC (1650) | 8644 ± 1033 | 10605 + 2259 |
| hUTC (825) | 10114 ± 676 | 10963 + 1929 |

Cells isolated from porcine urethra exhibited faster proliferation rates after three and seven days of co-culture with hUTC than when incubated in the basal medium (EGM-2/Hayflick). The rate of proliferation was dependent on the amount of hUTC present in the transwell. The effect was most pronounced at seven days of culture. The greatest effect was noticed with 6600 cells/well of hUTC, which produced a 35% increase in the proliferation rate of urethra-derived cells after seven days in culture.

### Conclusion

The above-presented data clearly indicates that hUTC have a positive in vitro effect on the proliferation rate of porcine urethra-derived cells. This suggests that at least partially, the mechanism of action of these cells responsible for restoration of leak point pressure (LPP) in incontinent rats (presented in Example 1), is increase in healthy cells and therefore regeneration of urethral tissue. This also suggests that their therapeutic effect is not just a bulking action but rather a trophic effect, which promotes bona fide long-term regenerative response.

We claim:

1. A composition for the treatment of incontinence comprising human umbilical tissue-derived cells and a carrier.

2. The composition of claim 1 wherein the human umbilical tissue-derived cells is allogeneic.

3. The composition of claim 1 wherein the carrier is selected from the group consisting of physiological buffer solution, injectable gel solution, saline and water.

4. The composition of claim 3 wherein the carrier is physiological buffer solution.

5. The composition of claim 4 wherein the physiological buffer solution is buffered saline, phosphate buffer solution, Hank's balanced salts solution, Tris buffered saline and Hepes buffered saline.

6. The composition of claim 3 wherein the carrier is an injectable gel solution comprising a physiological buffer and a gelling material.

7. The composition of claim 6 wherein the gelling material is selected from the group consisting of proteins, polysaccharides, polynucleotides, alginate, cross-linked alginate, poly(N-isopropylacrylamide), poly(oxyalkylene), copolymers of poly(ethylene oxide)-poly(propylene oxide), poly(vinyl alcohol), polyacrylate, monostearoyl glycerol co-Succinate/polyethylene glycol (MGSA/PEG) copolymers and combinations thereof.

8. The composition of claim 1 further comprising at least one microparticle.

9. The composition of claim 8 wherein the microparticle is comprised of a biocompatible polymer selected from the group consisting of synthetic polymers, natural polymers and combinations thereof.

10. A method of treating incontinence comprising injecting into a urogenital tissue the composition of claim 1.

11. A method of treating incontinence comprising injecting into a colorectal tissue the composition of claim 1.
